(19) United States
(12) Patent Application Publication
(10) Pub. No.: US 2005/0112160 A1
(43) Pub. Date: May 26, 2005

(54) Method of providing a blended composition

(75) Inventors: Paul Robert Tanner, Lebanon, OH (US); Larry Richard Robinson, Loveland, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/977,816

(22) Filed: Oct. 29, 2004

Related U.S. Application Data
(60) Provisional application No. 60/516,523, filed on Oct. 31, 2003.

(51) Int. Cl. A61K 7/00

(52) U.S. Cl. 424/401

(57) ABSTRACT

A method to provide a skin composition comprising at least one salt-form active which is aesthetically pleasing and effective. The method has the steps of providing a first container for a first aqueous phase having at least one salt-sensitive thickener; a second container for a second aqueous phase having at least one salt-form active; and dispensing the first and second phases from their respective containers onto a common surface. The first and second phases are then blended together and applied to the area of the skin in need of treatment.

### CROSS REFERENCE TO RELATED APPLICATION(S)

This application claims the benefit of U.S. Provisional Application No. 60/516,523, filed Oct. 31, 2003.

### FIELD OF THE INVENTION

The present invention relates to a method of providing to the skin a blended composition containing at least one salt-form active and at least one salt-sensitive thickener.

### BACKGROUND OF THE INVENTION

Many topically applied products currently available to consumers are directed primarily to improving the health and/or physical appearance of the skin. Many of these skin care products are directed to delaying, minimizing or even eliminating skin wrinkling, skin discolorations, and other histological changes typically associated with the aging and, or environmental damage to human skin.

Consumers prefer topically applied products since they are not only effective, but also safe and pleasant to use. Upon application of such topical products, they should not run or be sticky. Thus when the products are in the form of milks, lotions, creams or gels and the like, thickeners become an essential ingredient.

Recent research has resulted in the discovery that some of the most effective skin care actives are in the form of a salt. This creates a problem since many of the best thickeners that provide superior skin feel benefits are salt-sensitive. That is, in the presence of salts, these thickeners lose a significant portion of their ability to effectively thicken the topical products. As a result, the products are runny and messy. Increasing the level of such thickeners may improve the viscosity but results in inferior skin feel and decreased perception of absorption of the product into the skin.

An alternative to avoid the problems mentioned above is to use salt-insensitive thickeners in the composition. Salt-insensitive thickeners, however, typically do not exhibit good skin feel and application aesthetics whereby using them in topical compositions results in greasy and sticky feeling compositions that are not perceived as being absorbed well by the skin.

### SUMMARY OF THE INVENTION

Surprisingly, applicants have found a method to provide to the skin a composition comprising at least one salt-form active which is aesthetically pleasing and effective. The method has the following steps: a) providing a first container holding a first aqueous phase with at least one salt-sensitive thickener; b) providing a second container holding a second aqueous phase with at least one salt-form active; c) dispensing the first and second compositions from their respective containers onto a common surface, preferably skin and d) blending the first and second compositions together, forming a blended composition.

Both the first and second aqueous phases are, of course, water based. These aqueous phases have viscosities of from 100 to 1,000,000 centipoises. The phases can be solutions, dispersions of solids in water, oil-in-water emulsions, water-in-oil-in-water emulsions, and combinations of these.

In a preferred embodiment, the method has the following steps: a) providing a unitary container having a first compartment and a second compartment; b) providing a first aqueous phase comprising at least one salt-sensitive thickener in the first compartment; c) providing a second aqueous phase comprising the salt-form active(s) in the second compartment; d) dispensing the first and second phases from their respective compartments onto a common surface; and e) blending the first and second phases together to form a blended composition.

When the first and second aqueous phases are co-dispensed they can be blended in a number of ways. Firstly, they can be commingled upon contact with the common surface, such as skin. Alternatively, they are be blended either as the phases exit the discharge orifice or prior to the discharge orifice, for example, by means of the discharge orifice comprising a static mixer.

The invention further relates to an article of manufacture which is a unitary container with a first compartment containing a first phase with at least one salt-sensitive thickener and a second compartment containing a second phase with at least one salt-form active. The unitary container has a dispensing orifice wherein said first and said second phases are dispensed together. The dispensing orifice can include a static mixer.

### DETAILED DESCRIPTION OF THE INVENTION

While the specification concludes with the claims particularly pointing and distinctly claiming the invention, it is believed that the present invention will be better understood from the following description.

Definitions

As used herein, the “skin care products” are those used to treat or care for, or somehow moisturize, improve, protect, or clean the skin. Products contemplated by the phrase “skin care products” include, but are not limited to moisturizers, anti-aging products, personal cleansing products, anti-acne products, pharmaceuticals, cosmetics and the like.

The term “ambient conditions” as used herein refers to surrounding conditions under about one atmosphere of pressure, at about 50% relative humidity, and at about 25° C. unless otherwise specified.

The compositions of the present invention can include, consist essentially of, or consist of, the components of the present invention as well as other ingredients described herein. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the skin care compositions of the present invention, unless otherwise specified. All such weights as they pertain to listed ingredients are based on the active level and, therefore, do not include carriers or by-products that may be included in commercially available materials, unless otherwise specified.

The term “dermatologically-acceptable,” as used herein, means that the compositions or components thereof so described are suitable for use in contact with mammalian keratinous tissue without undue toxicity, incompatibility, instability, allergic response, and the like.

The term “safe and effective amount” as used herein means an amount of a compound or composition sufficient to significantly induce a positive benefit, preferably a positive keratinous tissue appearance or feel benefit, including independently or in combinations the benefits disclosed herein, but low enough to avoid serious side effects, i.e., to provide a reasonable benefit to risk ratio, within the scope of sound judgment of the skilled artisan.

The term “thickened topical composition”, as used herein, means a topical composition of from 100 centipoises to 1,000,000 centipoises, the viscosity having been increased by means of the addition of thickeners. These viscosity measurements are made by conventional means at ambient temperatures.

The term “skin care active”, as used herein, means any component of a topical composition that improves or protects the health and appearance of the skin.

The term “salt-sensitive thickener” as used herein means a thickener, which, in the presence of salt, loses a significant portion of its ability to thicken. When salt is added to a composition thickened with a salt-sensitive thickener, the composition “thins” dramatically even approaching a watery consistency.

The term “salt-insensitive thickener” as used herein means a thickener, which does not lose a significant portion of its ability to thicken in the presence of salt.

All percentages and ratios used herein are by weight of the total composition and all measurements made are at 25° C., unless otherwise designated.

As discussed in the summary above, the compositions in the claimed method comprise salt-sensitive thickeners and salt skin care actives. The composition used in the clamed invention comprise a first aqueous phase comprising at least one salt-sensitive thickener, preferably from about 0.01% to about 10%, more preferably from about 0.05% to about 5%, and even more preferably from about 0.25% to about 4%, by weight of the first aqueous phase.

Generally, salt-sensitive thickeners are polymers that contain ionizable groups and that are able to more effectively thicken water or water-based systems when all or a portion of these groups are ionized. Non-limiting examples of types of salt-sensitive thickeners include the following:

i) Carboxylic Acid Polymers

These polymers are compounds containing one or more monomers with an ionizable carboxylic acid group, such as monomers derived from acrylic acid, substituted acrylic acids, and salts of these acrylic acids and the substituted acrylic acids.

Examples of commercially available carboxylic acid polymers useful herein include the carbomers, which are homopolymers of acrylic acid crosslinked with allyl ethers of sucrose or pentaerytritol. Examples of carbomers are the Carbopol® 900 series from Noveon (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen® TR-1, and Pemulen® TR-2, and Ultrez-21® from Noveon. Also available are sodium acrylate copolymers, such as Luvigel®EM from BASF, Salcare SC-91® from Ciba Specialty Chemicals Corporation, and acrylate/acrylamide copolymers, such as Polymer EX-617 from Noveon.

ii) Cationic Polymers

The first aqueous phase of the present invention can optionally comprise cationic polymeric thickening agent, including cationic crosslinked polyacrylate polymers. Examples of useful cationic polymers are polyquaternium-32, available as Salcare®SC-92, and polyquaternium-37 available as Salcare®SC-95 and SC-96, all from Ciba Specialty Chemicals Corporation. Additional cationic polymers are those described in U.S. Pat. No. 5,100,660, U.S. Pat. No. 4,849,484, U.S. Pat. No. 4,835,206, U.S. Pat. No. 4,628,078, U.S. Pat. No. 4,599,379, and EP 228,868.

iii) Sulfonated Polymers

The first aqueous phase of the present invention can optionally comprise polymers containing one or more monomers containing a sulfonic acid or sulfonate group. Examples available from Clariant GmbH include: ammonium acryloyldimethyl taurate/VP copolymer; and ammonium polyacryloyldimethyl taurate. SEPPIC produces suitable polymers under the Simulgel® tradename, such as, hydroxyethyl acrylate/sodium acryloyldimethyl taurate copolymer; sodium acrylate/acryloyldimethyl taurate copolymer; sodium polyacryloyldimethy taurate, and acrylamide/sodium acryloyldimethyl taurate copolymer.

Although not necessary, a second aqueous phase of the compositions used in the present invention may comprises one or more salt-insensitive thickening agents. If used at all, the total amount salt insensitive thickener in the second aqueous phase is from about 0.01% to about 10%. preferably from about 0.1% to about 6%, by weight. Nonlimiting examples of type of salt-insensitive thickeners include the following:

i) Polysaccharides

A wide variety of polysaccharides are useful herein. “Polysaccharides” refer to gelling agents that contain a backbone of repeating sugar (i.e., carbohydrate) units. Nonlimiting examples of polysaccharide gelling agents include those selected from the group consisting of cellulose, hydroxyethylcellulose, hydroxyethyl ethylcellulose, hydroxypropylcellulose, hydroxypropyl methylcellulose, methyl hydroxyethylcellulose, microcrystalline cellulose, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation.

Other useful polysaccharides include scleroglucans comprising a linear chain of (1-3) linked glucose units with a (1-6) linked glucose every three units, a commercially available example of which is Clearogel™ CS11 from Michel Mercier Products Inc.

ii) Gums

Other salt-insensitive thickening and gelling agents useful herein include materials that are primarily derived from natural sources, such as natural gums. Nonlimiting examples of natural gums include guar gums, locust bean gum, xanthan gum and mixtures thereof.

iii) Starch and Starch Derivatives.

Other salt-insensitive thickeners useful herein, include starches and starch derivatives. Nonlimiting examples include Structure Solance® a modified potato starch, and Structure XL®, a hydroxypropyl starch phosphate, both commercially available from National Starch.

Another essential element of the compositions used in the claimed invention is a skin care active, specifically those in their salt form. For purposes of this application, salt-form actives dissociate in an aqueous solution to their respective positively and negatively charged components.

The composition used in the present invention includes salt-form skin care actives, preferably from 0.01% to 50%, more preferably from about 0.05% to 25% and even more preferably from 0.1% to 10% by weight of the second aqueous phase. These salt-form skin care actives are selected from the group consisting of inorganic and organic salts that at least partially dissociate in an aqueous environment.

1. Inorganic Salts

The compositions of the present invention optionally include a safe and effective amount of an inorganic salt. Inorganic salts are characterized as not containing a hydrogen that can be displaced. An example of an inorganic salt is zinc pyrithione (ZPT).

2. Organic Salts

The compositions used in the present invention optionally include a safe and effective amount of an organic salt. Organic salts are characterized as formed from reacting an acid containing a hydrogen that can be displaced with a base.

a) Carboxylic Acid Salts

The carboxylic acid salts and derivatives thereof of the present invention correspond to the formula:
RCO₂X
wherein X is Na, K, Mg, Mn, Zn, Cu, triethanolamine, diethanolamine, ammonium, quaternary alkyl ammonium); R is C1-C20 straight or branched alkyl or aryl groups. As used herein, alkyl means carbon containing chains that may be straight or branched or cyclic, substituted or unsubstituted, saturated or monounsaturated or polyunsaturated.

Examples include salts of hydroxy acids (e.g., salicylic acid, glycolic acid, lactic acid, 3-hydroxy benzoic acid, 4-hydroxy benzoic acid, 2-hydroxybutanoic acid, 2-hydroxypentanoic acid), 2-hydroxyhexanoic acid keto acids (e.g., pyruvic acid), phytic acid, glycyrrhetic acid, butylated hydroxy benzoic acids, cis-retinoic acid, trans-retinoic acid, phytic acid, lipoic acid, azelaic acid, arachidonic acid, lysophosphatidic acid, salts of sugar acids (e.g., Glucosamine Hydrochloride, Mn gluconate) and salts of amino acids (e.g. Undecenoyl Phenylalanine, Dipalmitoyl Hydroxyproline) and mixtures thereof.

b) Non-Carboxylic Acid Salts

There are a number of acids that are not carboxylic acids that contain an acidic hydrogen that is capable of being displaced by an base that are also applicable in this invention. Included among such non-carboxylic acid salts is sodium dehydroacetate.

c) Sulfonic Acid Salts

The sulfonic acid salts useful in the present invention correspond to the formula:
RSO₃X
wherein X is metal selected from the group consisting of Na, K, Mg, Mn, Zn, Cu; triethanolamine, diethanolamine, ammonium, quaternary alkyl ammonium, R is a C1-C20 straight or branched alkyl or aryl. Alkyl means carbon containing chains that may be straight or branched or cyclic, substituted or unsubstituted, saturated or monounsaturated or polyunsaturated.

Examples are sodium or triethanolamine salts of 2-phenylbenzimidazole-5-sulfonic acid, and the triethanolamine salt of 2-hydroxy-4-methoxybenzophenone-5-sulfonic acid.

d) Phosphoric Acid Salts

The phosphoric acid salt of the present invention corresponds to the formula:
ROPO₃X
wherein X is Na, K, Mg, Mn, Zn, Cu, triethanolamine, diethanolamine, ammonium, quaternary alkyl ammonium); R is C1-C20 straight or branched alkyl or aryl groups. As used herein, alkyl means carbon containing chains that may be straight or branched or cyclic, substituted or unsubstituted, saturated or monounsaturated or polyunsaturated.

Preferred examples are magnesium ascorbyl phosphate and sodium ascorbyl phosphate.

e) Amine Salts

Amine salts formed by reacting an acid with an amine to form a quaternized amine are claimed. Examples are salts of vitamin B compounds (e.g., thiamine (vitamin B1), pantothenic acid (vitamin B5), Pryidoxine (vitamin B6), arachidonic acid, linoleic acid, linolenic acid (Vitamin F), riboflavin (vitamin B2).

The compositions used in the present invention may contain other skin care actives that are conventionally used in their non-salt form. When incorporated into the composition these actives are suitable for use in contact with human keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical materials commonly used in the skin care industry, that are suitable for use in the compositions used in the present invention. Examples of these classes of materials include: abrasives, absorbents, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), skin bleaching and lightening agents, skin-conditioning agents, skin soothing and/or healing agents and derivatives, skin treating agents, and vitamins and derivatives thereof. Preferred skin care actives are N-acyl amino acids and their isomers, sugar amines, vitamin B₃, retinoids, peptides, hexamidine and its derivatives, salicylic acid and phytosterol.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

1. Desquamation Actives

A safe and effective amount of a desquamation active may be added to the compositions of the present invention, more preferably from about 0.01% to about 10%, even more preferably from about 0.5% to about 5%, also preferably from about 0.1% to about 2%, by weight of the composition. Desquamation actives enhance the skin appearance benefits of the present invention. For example, the desquamation actives tend to improve the texture of the skin (e.g., smoothness). One desquamation system that is suitable for use herein comprises salicylic acid and zwitterionic surfactants and is described in U.S. Pat. No. 5,652,228. Zwitterionic surfactants such as described in these applications are also useful as desquamatory agents herein, with lonzaine being particularly preferred.

2. Anti-Acne Actives

The compositions of the present invention may comprise a safe and effective amount of one or more anti-acne actives. Examples of useful anti-acne actives include resorcinol, sulfur, erythromycin, zinc, dehydroacetic acid, etc. Further examples of suitable anti-acne actives are described in further detail in U.S. Pat. No. 5,607,980.

3. Anti-Wrinkle Actives/Anti-Atrophy Actives

The compositions of the present invention may further comprise a safe and effective amount of one or more anti-wrinkle actives or anti-atrophy actives. Exemplary anti-wrinkle/anti-atrophy actives suitable for use in the compositions of the present invention include hydroxy acids (e.g., salicylic acid, glycolic acid), keto acids (e.g., pyruvic acid), ascorbic acid (vitamin C), phytic acid, lysophosphatidic acid, flavonoids (e.g., isoflavones, flavones, etc.), stilbenes, cinnamates, resveratrol, kinetin, zeatin, dimethylaminoethanol, peptides from natural sources (e.g., soy peptides), and retinoids which enhance the keratinous tissue appearance benefits of the present invention, especially in regulating keratinous tissue condition, e.g., skin condition, and other vitamin B compounds (e.g., thiamine (vitamin B1), pantothenic acid (vitamin B5), carnitine (vitamin Bt), riboflavin (vitamin B2), and their derivatives.

4. Anti-Oxidants/Radical Scavengers

The compositions of the present invention may include a safe and effective amount of an anti-oxidant/radical scavenger. The anti-oxidant/radical scavenger is especially useful for providing protection against UV radiation that can cause increased scaling or texture changes in the stratum corneum and against other environmental agents, which can cause skin damage.

A safe and effective amount of an anti-oxidant/radical scavenger may be added to the compositions of the subject invention, preferably from about 0.01% to about 10%, more preferably from about 0.1% to about 5%, of the composition.

Anti-oxidants/radical scavengers such as ascorbic acid (vitamin C), ascorbyl esters of fatty acids, ascorbic acid derivatives, tocopherol (vitamin E), tocopherol sorbate, tocopherol acetate, other esters of tocopherol, butylated hydroxy benzoic acids, 6-hydroxy-2,5,7,8-tetramethylchroman-2-carboxylic acid (commercially available under the tradename Trolox®), amines (e.g., N,N-diethylhydroxylamine, amino-guanidine), nordihydroguaiaretic acid, bioflavonoids, amino acidssilymarin, tea extracts, and grape skin/seed extracts may be used. Preferred anti-oxidants/radical scavengers are selected from esters of tocopherol, more preferably tocopherol acetate.

5. Chelators

The compositions of the present invention may also comprise a safe and effective amount of a chelator or chelating agent. As used herein, “chelator” or “chelating agent” means an active agent capable of removing a metal ion from a system by forming a complex so that the metal ion cannot readily participate in or catalyze oxygen radical formation. The inclusion of a chelating agent is especially useful for providing protection against UV radiation that can contribute to skin damage.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884. Preferred chelators useful in compositions of the subject invention are furildioxime and derivatives thereof.

6. Flavonoids

The compositions of the present invention may optionally comprise a flavonoid compound. Flavonoids are broadly disclosed in U.S. Pat. Nos. 5,686,082 and 5,686,367. Examples of flavonoids particularly suitable for use in the present invention are one or more flavones, one or more isoflavones, one or more coumarins, one or more chromones, one or more dicoumarols, one or more chromanones, one or more chromanols, isomers (e.g., cis/trans isomers) thereof, and mixtures thereof.

Preferred for use herein are flavones and isoflavones, in particular daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy), and mixtures thereof.

Flavonoid compounds useful herein are commercially available from a number of sources, e.g., Indofine Chemical Company, Inc., Steraloids, Inc., and Aldrich Chemical Company, Inc.

The herein described flavonoid compounds are preferably present in the instant invention at concentrations of from about 0.01% to about 20%, more preferably from about 0.1% to about 10%, and even more preferably from about 0.5% to about 5%.

7. Anti-Inflammatory Agents

A safe and effective amount of an anti-inflammatory agent may be added to the compositions of the present invention, preferably from about 0.01% to about 10%, more preferably from about 0.5% to about 5%, of the composition. The anti-inflammatory agent enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency.

Steroidal anti-inflammatory agents, include but are not limited to, corticosteroids such as hydrocortisone. A second class of anti-inflammatory agents, which is useful in the compositions, includes the nonsteroidal anti-inflammatory agents. The varieties of compounds encompassed by this group are well known to those skilled in the art. Specific non-steroidal anti-inflammatory agents useful in the composition invention include, but are not limited to, salicylates, flufenamic acid, etofenamate, aspirin, and mixtures thereof.

Additional anti-inflammatory agents useful herein include allantoin and compounds of the Licorice (the plant genus/species Glycyrrhiza glabra) family, including glycyrrhetic acid, glycyrrhizic acid, and derivatives thereof (e.g., esters).

8. Anti-Cellulite Agents

The compositions of the present invention may also comprise a safe and effective amount of an anti-cellulite agent. Suitable agents may include, but are not limited to, xanthine compounds (e.g., caffeine, theophylline, theobromine, and aminophylline).

8. Tanning Actives

The compositions of the present invention may comprise a tanning active. When present, it is preferable that the compositions comprise from about 0.1% to about 20%, more preferably from about 2% to about 7%, and even more preferably from about 3% to about 6%, by weight of the composition, of a tanning active. A preferred tanning active is dihydroxyacetone.

9. Skin Lightening Agents

The compositions of the present invention may comprise a skin lightening agent. When used, the compositions preferably comprise from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, tranexamic acid, ascorbic acid and derivatives thereof (e.g., ascorbyl glucoside, and the like). Other skin lightening materials suitable for use herein include Acitwhite® (Cognis), Emblica® (Rona), Azeloglicina (Sinerga) and extracts (e.g. mulberry extract).

10. Antimicrobial and Antifungal Actives

The compositions of the present invention may comprise an antimicrobial or antifungal active. Such actives are capable of destroying microbes, preventing the development of microbes or preventing the pathogenic action of microbes. A safe and effective amount of an antimicrobial or antifungal active may be added to the present compositions, preferably, from about 0.001% to about 10%, more preferably from about 0.01% to about 5%, and even more preferably from about 0.05% to about 2% by weight of the composition.

Preferred examples of actives useful herein include those selected from the group consisting of salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, ciclopirox, lidocaine hydrochloride, clotrimazole, miconazole, ketoconazole, neocycin sulfate, and mixtures thereof.

11. Sunscreen Actives

The compositions of the subject invention may optionally contain a sunscreen active. As used herein, “sunscreen active” includes both sunscreen agents and physical sunblocks. Suitable sunscreen actives may be organic or inorganic.

A wide variety of conventional sunscreen actives are suitable for use herein. Sagarin, et al., at Chapter VIII, pages 189 et seq., of Cosmetics Science and Technology (1972), discloses numerous suitable actives. Particularly suitable sunscreen agents are 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds.

Preferred organic sunscreen actives useful in the compositions useful in the subject invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, zinc oxide, titanium dioxide, and mixtures thereof. Especially preferred sunscreen actives include 4,4′-t-butylmethoxydibenzoylmethane, 2-ethylhexyl-p-methoxycinnamate, phenyl benzimidazole sulfonic acid, octocrylene, zinc oxide, and titanium dioxide, and mixtures thereof.

A safe and effective amount of the sunscreen active is used, typically from about 1% to about 20%, more typically from about 2% to about 10% by weight of the composition. Exact amounts will vary depending upon the sunscreen chosen and the desired Sun Protection Factor (SPF).

12. Conditioning Agents

The compositions of the present invention may comprise a conditioning agent selected from the group consisting of humectants, moisturizers, or skin conditioners, including emollients. A variety of these materials can be employed and each can be present at a level of from about 0.01% to about 40%, more preferably from about 0.1% to about 30%, and even more preferably from about 0.5% to about 25% by weight of the composition. These materials include, but are not limited to, guanidine; urea; glycolic acid; lactic acid; aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; sucrose polyester; petrolatum; silicones, silicone elastomers, hydrocarbon oils, fatty alcohols, fatty acids, esters of mono and dibasic carboxylic acids with mono and polyhydric alcohols, polyoxyethylenes, polyoxypropylenes; mixtures of polyoxyethylene and polyoxypropylene ethers of fatty alcohols and mixtures thereof.

Silicones useful in the composition herein include polyalkylsiloxanes with viscosities of from about 0.5 to about 1,000,000 centistokes at 25° C. Commercially available polyalkylsiloxanes include the polydimethylsiloxanes, which are also known as dimethicones, examples of which include the Vicasil® series sold by General Electric Company and the Dow Corning® 200 series sold by Dow Corning Corporation. Cyclic polyalkylsiloxanes suitable for use in the composition include those commercially available such as Dow Corning® 244, Dow Corning® 344 fluid, and Dow Corning® 345 fluid.

Suitable for use herein are silicone elastomers, which can be emulsifying or non-emulsifying crosslinked siloxane elastomers or mixtures thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Emulsifying crosslinked organopolysiloxane elastomers can notably be chosen from the crosslinked polymers described in U.S. Pat. Nos. 5,412,004, 5,837,793, and 5,811,487.

Advantageously, the non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. No. 4,970,252, U.S. Pat. No. 5,760,116, and U.S. Pat. No. 5,654,362. Additional crosslinked organopolysiloxane elastomers useful in the present invention are disclosed in Japanese Patent Application JP 61-18708, assigned to Pola Kasei Kogyo KK.

Preferably, the conditioning agent is selected from the group consisting of glycerol, urea, petrolatum, sucrose polyester, silicones, silicone elastomers, esters, and combinations thereof.

13. Water-Soluble Vitamins

The compositions of the present invention may contain a safe and effective amount of one or more water-soluble vitamins. Examples of water-soluble vitamins include, but are not limited to, water-soluble versions of vitamin B, vitamin B derivatives, vitamin C, vitamin C derivatives, vitamin K, vitamin K derivatives, vitamin D, vitamin D derivatives, vitamin E, vitamin E derivatives, and mixtures thereof. The vitamin compounds may be included as the substantially pure material, or as an extract obtained by suitable physical and/or chemical isolation from natural (e.g., plant) sources. When vitamin compounds are present in the compositions of the instant invention, the compositions preferably contain from about 0.0001% to about 50%, more preferably from about 0.001% to about 10%, still more preferably from about 0.01% to about 5%, and still more preferably from about 0.1% to about 5%, by weight of the composition, of the vitamin compound.

14. Particulate Material

The compositions of the present invention may contain one or more particulate materials. Nonlimiting examples of particulate materials useful in the present invention include colored and uncolored pigments, interference pigments, inorganic powders, organic powders, composite powders, optical brightener particles, and combinations thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion. These particulate materials may provide a wide range of functions, including but not limited to modifying skin feel, masking the appearance of certain skin characteristics such as blotchy areas, age spots, freckles, fine lines, wrinkles, and pores, absorbing excess skin sebum/oils, reducing skin shine, improving application properties of the composition, masking the color of other components of the composition, filling in skin pores, lines and wrinkles, and reducing migration of liquid materials on the skin. Preferably, particulate materials are present in the composition in levels of from about 0.01% to about 20%, more preferably from about 0.05% to about 10%, still more preferably from about 0.1% to about 5%, by weight of the composition. There are no specific limitations as to the pigment, colorant or filler powders used in the composition.

Particulate materials useful herein include but are not limited to bismuth oxychloride, sericite, mica, mica treated with barium sulfate or other materials, zeolite, kaolin, silica, boron nitride, lauroyl lysine, nylon, polyethylene, talc, styrene, polypropylene, polystyrene, ethylene/acrylic acid copolymer, sericite, aluminum oxide, silicone resin, barium sulfate, calcium carbonate, cellulose acetate, PTFE, polymethyl methacrylate, starch, modified starches such as aluminun starch octenyl succinate, silk, glass, and mixtures thereof. Preferred organic powders/fillers include, but are not limited, to polymeric particles chosen from the methylsilsesquioxane resin microspheres such as for example those sold by Toshiba silicone under the name Tospearl 145A; microspheres of polymethylmethacrylates such as those sold by Seppic under the name Micropearl M 100; the spherical particles of crosslinked polydimethylsiloxanes, especially such as those sold by Dow Corning Toray Silicone under the name Trefil E 506C or Trefil E 505C, sphericle particles of polyamide and more specifically Nylon 12, especially such as those sold by Atochem under the name Orgasol 2002D Nat C05, polystyerene microspheres such as for example those sold by Dyno Particles under the name Dynospheres, ethylene acrylate copolymer sold by Kobo under the name FloBead EA209, PTFE, polypropylene, aluminium starch ocetenylsuccinate such as those sold by National Starch under the name Dry Flo, microspheres of polyethylene such as those sold by Equistar under the name of Microthene FN510-00, silicone resin, polymethylsilsesquioxane silicone polymer, platelet shaped powder made from L-lauroyl lysine, and mixtures thereof. Especially preferred are spherical powders with an average primary particle size from 0.1 to 75 microns, preferably from 0.2 to 30 microns.

Also useful herein are interference pigments. Interference pigments, for purposes of the present specification are defined as thin platelike layered particles having two or more layers of controlled thickness with different refractive indices that yield a characteristic reflected color from the interference of typically two, but occasionally more, light reflections, form different layers of the platelike particle. The most common examples of interference pigments are micas layered with about 50-300 nm films of TiO2, Fe2O3, silica, tin oxide, and/or Cr2O3. Such pigments are often peralescent. Pearl pigments reflect, refract and transmit light because of the transparency of pigment particles and the large difference in the refractive index of mica platelets and, for example, the titanium dioxide coating. Useful intereference pigments are available commercially from a wide variety of suppliers, for example, Rona (Timiron™ and Dichrona™), Presperse (Flonac™), Englehard (Duochrome™), Kobo (SK45-R and SK-45-G), BASF (Sicopearls) and Eckart (e.g. Prestige Silk Red). Especially preferred are interference pigments with smaller particle sizes, with an average diameter of individual particles less than about 75 microns in the longest direction, preferably with an average diameter less than about 50 microns.

Other pigments useful in the present invention provide color primarily through selective absorption of specific wavelengths of visible light, and include inorganic pigments, organic pigments and combinations thereof. Examples of useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO2, ZnO, or ZrO2, are commercially available from a number of sources. One example of a suitable particulate material contains the material available from U.S. Cosmetics (TRONOX TiO2 series, SAT-T CR837, a rutile TiO2). Particularly preferred are charged dispersions of titanium dioxide, as are disclosed in U.S. Pat. No. 5,997,887.

Preferred colored or uncolored non-interference-type pigments have a primary average particle size of from about 10 nm to about 100,000 nm, more preferably from about 20 nm to about 5,000 nm, even more preferably from about 20 nm to about 1000 nm. Mixtures of the same or different pigment/powder having different particle sizes are also useful herein (e.g., incorporating a TiO2 having a primary particle size of from about 100 nm to about 400 nm with a TiO2 having a primary particle size of from about 10 nm to about 50 nm).

The pigments/powders of the current invention can be surface treated to provide added stability of color and/or for ease of formulation. Nonlimiting examples of suitable coating materials include silicones, lecithin, amino acids, metal soaps, polyethylene and collagen. These surface treatments may be hydrophobic or hydrophilic, with hydrophobically treatments being preferred. Particularly useful hydrophobic pigment treatments include polysiloxane treatments such as those disclosed in U.S. Pat. No. 5,143,722.

In addition to the skin care actives disclosed above, the compositions disclosed herein can comprise other components including but not limited to aesthetic components such as fragrances, opacifying agents, pH adjusters, reducing agents, sun-less tanning agents, preservatives, emulsifiers and sequestrants.

Emulsifiers useful herein are well known in the art, and include nonionic, anionic, cationic, and amphoteric emulsifiers. Non-limiting examples of emulsifiers useful in the oil-in-water emulsions of this invention are given in McCutcheon's, Detergents and Emulsifiers, North American Edition (1986), published by Allured Publishing Corporation; U.S. Pat. No. 5,011,681; U.S. Pat. No. 4,421,769; and U.S. Pat. No. 3,755,560.

Packaging

As discussed above, the compositions used in the present invention may utilize multiple containers to separate the salt-form active from the salt sensitive thickeners. Optionally a unitary container such as that available from Airspray International B.V. (Netherlands) can be used. The unitary container offers convenience and simplified use having a single orifice wherein said first and second phases are dispensed together. Such a container is disclosed in U.S. Pat. No. 6,220,483. This orifice may also comprise a static mixer. Alternate designs, which result in the dispensing characteristics required herein are also acceptable. Alternate embodiments can include tubes, single- and multiple-use sachets, and dual compartment pouches.

In a further embodiment the invention can be a kit having a first container holding a first aqueous phase comprising at least one salt-sensitive thickener and second container holding a second aqueous phase comprising at least one salt-form active. Both the first and second containers can comprise a dispenser and a set of instructions for dispensing the first and second compositions, then blending them and applying the blended composition.

### Composition Preparation

Each phase of the topical compositions of the present invention are generally prepared by conventional methods such as are known in the art of making topical compositions. Such methods typically involve mixing of the ingredients in one or more steps to a relatively uniform state, with or without heating, milling, cooling, application of vacuum, and the like. The compositions are preferably prepared such as to optimize stability (physical stability, chemical stability, photostability) and/or delivery of the active materials (e.g., sodium dehydroacetate, N-acyl amino acids, sugar amine, vitamin B₃, retinoid, phytosterol, dialkanoyl hydroxyproline, hexamidine, salicylic acid). This optimization may include appropriate pH (e.g., less than 8), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

A wide range of quantities of the compositions of the present invention can be employed to provide a skin appearance and/or feel benefit. Quantities of the present compositions, which are typically applied per application are, in mg composition/cm² skin, from about 0.01 mg/cm² to about 20 mg/cm². A particularly useful application amount is about 0.1 mg/cm² to about 5 mg/cm².

### EXAMPLES

The following examples further describe and demonstrate the embodiments within the scope of the present invention. The examples are given solely for the purpose of illustration and are not to be construed as limitations of the present invention, as many variations thereof are possible without departing from the spirit and scope of the invention.

### Examples A1, A2, and A3

The following are examples of the aqueous phase containing the salt sensitive thickener and are made by conventional methods

| Example: | A1 | A2 | A3 |
|---|---|---|---|
| Ingredient: | Weight % | Weight % | Weight % |
| Water | QS to 100% | QS to 100% | QS to 100% |
| Glycerin | 5 | 10 | 7 |
| Butylene Glycol | 2 | — | — |
| Aloe Vera Gel | — | 0.1 | 0.1 |
| Grapeseed Extract | — | — | 0.01 |
| Green Tea Extract | — | 0.5 | 0.1 |
| Glydant Plus Liquid | 0.3 | 0.3 | 0.3 |
| N-Acetyl Glucosamine | — | 0.5 | 2 |
| Niacinamide | 3 | 5 | 4 |
| Dex-Panthenol | 0.5 | 1 | 0.5 |
| Disodium EDTA | 0.05 | 0.05 | 0.1 |
| Polysorbate 20 | 0.3 | 0.5 | — |
| Laureth-4 | 0.1 | — | 0.1 |
| Cyclomethicone D5 | 10 | 14 | 10 |
| Dimethicone | — | — | 3 |
| Isohexadecane | 3 | — | — |
| Phenyl Trimethicone | — | 2 | — |
| Dow Corning 9045¹ | — | — | 8 |
| USG-103² | — | 4 | — |
| Dow Corning 1503³ | — | 0.5 | 2 |
| Polymethylsilsesquioxane | — | 2 | — |
| Microthene FN510-00⁴ | — | — | 1 |
| Titanium Dioxide | 0.25 | — | — |
| Prestige Silk Red⁵ | — | — | 1 |
| Simulgel EG⁶ | 3 | — | — |
| Carbopol Ultrez 21⁷ | — | 0.35 | — |
| Sepigel 305⁸ | — | — | 2 |
| Triethanolamine | — | 0.5 | — |
| Colored Dyes | 0.002 | — | — |
| Fragrance | 0.05 | — | 0.1 |

¹A silicone elastomer blend from Dow Corning Corporation
²A silicone elastomer blend from ShinEtsu
³A silicone gum blend from Dow Corning Corporation
⁴A spherical polyethylene powder from Equistar
⁵A layered mica/titanium dioxide/tin oxide interference pigment from Eckart
⁶A sodium acrylate/acryloyldimethyl taurate copolymer thickening agent from Seppic
⁷An acrylates/C10-30 alkyl acrylate crosspolymer from Noveon
⁸A polyacrylamde based copolymer (with some sulfonic acid functionality) from Seppic

### Examples B1, B2, and B3

The following are examples of the aqueous phase containing the salt form active.

| Example: | B1 | B2 | B3 |
|---|---|---|---|
| Ingredient: | Weight % | Weight % | Weight % |
| Water | QS to 100% | QS to 100% | QS to 100% |
| Glycerin | 10 | 2 | — |
| Phenyl Benzimidazole | 4 | — | — |
| Sulfonic acid | | | |
| Sodium Dehydroacetate | — | — | 6.0 |
| Undecylenoyl Phenylalanine | — | 5 | — |
| Triethanolamine | 2.6 | 2.8 | — |
| Hydroxypropyl Starch | 3.0 | — | 4.3 |
| Phosphate | | | |
| Xanthan Gum | — | 0.5 | — |
| Hydoxyethyl Cellulose | 0.75 | — | — |
| Liquapar Optima⁹ | — | — | 0.3 |
| Glydant Plus¹⁰ | 0.15 | 0.15 | — |
| Disodium EDTA | 0.01 | 0.05 | 0.05 |
| Colored Dyes | — | — | 0.01 |
| Fragrance | 0.02 | — | — |

⁹A preservative blend from ISP
¹⁰A preservative blend from Lonza

### Example 1

Fill one container with A1, and a second container with B1. Dispense 0.3 grams of A1 and 0.1 grams of B1. Mix the two materials together prior to rubbing into the skin to deliver the sunscreen phenyl benzimidazole sulfonic acid to the skin with good skin feel.

### Example 2

Fill one container with A2, and a second container with B2. Dispense 0.4 grams of A2 and 0.1 grams of B2. Mix the two materials together when rubbing onto the skin to deliver an effective amount of the skin active undecylenoyl phenylalanine to the skin with good skin feel.

### Example 3

Fill one compartment of a dual phase pump dispenser with A3, and a second compartment of the dual phase dispenser with B3. Simultaneously dispense 0.44 grams of A3 with 0.04 grams of B3. The two compositions dispense together. Rub this blend into the skin to deliver an effective level of the skin active sodium dehydroacetate to the skin with good skin feel.

### Example 4

Fill one container with A3, and a second container with B2. Dispense 0.5 grams of A3 and 0.02 grams of B2. Mix the two materials together prior to rubbing into the skin to deliver sodium dehydroacetate to the skin with good skin feel.

### Example 5

Fill one compartment of a dual phase tube dispenser with A1, and a second compartment of the dual phase dispenser with B2. Simultaneously dispense 0.44 grams of A1 with 0.04 grams of B2, The two compositions dispense together. Rub this blend into the skin.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method to provide to the skin a composition comprising at least one salt-form active said method comprising the steps of:
a) providing a first container holding a first aqueous phase comprising at least one salt-sensitive thickener;
b) providing a second container holding a second aqueous phase comprising said at least one salt-form active;
c) dispensing from their respective containers onto a common surface, said first composition and said second composition; and
d) blending said first and second compositions together, forming a blended composition.

2. A method to provide to the skin a composition comprising at least one salt-form active said method comprising the steps of:
a) providing a unitary container having a first compartment and a second compartment;
b) providing a first aqueous phase comprising at least one salt-sensitive thickener in said first compartment;
c) providing a second aqueous phase comprising said at least one salt-form active said second compartment;
d) dispensing from their respective compartments onto a common surface, said first phase and said second phase; and
e) blending said first and second phases together to form said composition.

3. The method of claim 2 wherein said common surface comprises the areas of mammalian skin in need of treatment with said composition.

4. The method of claim 2 wherein the weight ratio, as dispensed, of said first phase to said second phase is from 50:1 to 1:1.

5. The method of claim 2 wherein said container comprises a dispenser that co-dispenses said first aqueous phase and said second aqueous phase wherein said phases are commingled upon contacting said common surface.

6. The method of claim 2 wherein the step of blending of said first and second phases is accomplished as said phases exit the discharge orifice of said container.

7. The method of claim 6 wherein blending of said first and second phases is accomplished prior to the discharge orifice of said container.

8. The method of claim 2 wherein said at least one salt-form active is selected from the group consisting of inorganic salts, organic carboxylic acid salts, organic non-carboxylic acid salts, organic sulfonic acid salts, organic phosphonic acid salts, organic amine salts and mixtures thereof.

9. The method of claim 8 wherein said salt-form active is selected from the group consisting of sodium dehydroacetate, undecylenoyl phenylalanine, dipalmitoyl hydroxyproline, and mixtures thereof.

10. The method of claim 2 wherein the salt-sensitive thickeners are selected from the group consisting of carboxylic acid polymers, cationic polymers, sulfonic acid polymers and mixtures thereof.

12. The method of claim 2 wherein said first aqueous phase further comprises at least one non-salt active.

13. The method of claim 12 wherein said non-salt actives are selected from the group consisting of desquamation actives, anti-acne actives, anti-wrinkle actives, anti-atrophy actives, anti-oxidants, radical scavengers, chelators, flavonoids, anti-inflammatory agents, anti-cellulite agents, tanning actives, skin lightening agents, antimicrobial and antifungal actives, sunscreen actives, conditioning agents, water-soluble vitamins, particulate materials, and mixtures thereof.

14. The method of claim 2 wherein said second aqueous phase further comprises at least one salt-insensitive thickener.

15. The method of claim 14 wherein said salt-insensitive thickeners are selected from the group consisting of polysaccharides, gums, starch and starch derivatives, and mixtures thereof.

16. The method of claim 15 wherein said salt-insensitive thickener is a starch derivative.

17. An article of manufacture comprising a unitary container having at least a first compartment containing a first phase comprising at least one salt-sensitive thickener and a second compartment containing a second phase comprising at least one salt-form active, said container further comprising a dispensing orifice wherein said first and said second phases are dispensed together.

18. The article of manufacture according to claim 17 wherein said dispensing orifice comprising a static mixer.

19. A kit comprising a first container holding a first aqueous phase comprising at least one salt-sensitive thickener and second container holding a second aqueous phase comprising said at least one salt-form active, both first and second containers comprising a dispenser and a set of instructions for dispensing said first and second compositions, blending said first and second compositions and application of said blended first and second composition.

20. A kit comprising a unitary container having a first compartment holding a first aqueous phase comprising at least one salt-sensitive thickener and second compartment holding a second aqueous phase comprising said at least one salt-form active, said unitary container further comprising a dispenser wherein said first and second phases are dispensed together; said kit further comprising a set of instructions for dispensing said first and second phases, blending said first and second phases and application of said blended first and second phases.
